SEQUENCE LISTING (1 sequence)

SEQ ID NO: 1 - DNA, 1343 bp, Streptomyces sp.
  misc_feature (338)..(338): n is a, c, g, or t
  misc_feature (978)..(978): n is a, c, g, or t
  misc_feature (989)..(989): n is a, c, g, or t
  misc_feature (1084)..(1084): n is a, c, g, or t
agtttgatcctggctcaggacgaacgctggcggcgtgcttaacacatgcaagtcgaacgatgaaccactttcggtggggattagtggcgaacgggtgagtaacacgtgggcaatctgccctgcactctgggataaccccgggaaaccggggctaataccggatacgacccccgggggcatcctcgggggtggaaagctccggcggtgcaggatgagcccgcggcctatcagctggttggtgaggtaacggctcaccaaggcgacgacgggtagccggcctgagagggcgaccggccacactgggactgagacacggcccagactcctacgggaggcancagtggggaatattgcacaatgggcgcaagcctgatgcagcgacgccgcgtgagggatgacggccttcgggttgtaaacctctttcagcagggaagaagccgcgaggtgacggtacctgcagaagaagcgccggctaactacgtgccagcagccgcggtattacgtagggcgcgagcgttgtccggaattattgggcgtaaagagctcgtaggcggcttgtcgcgtcggttgtgaaagcccggggcttaaccccgggtctgcagtcgatacgggcaggctggagttcggcaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacggtgggcactaggtgtggggggcattccacgtcctccgtgccgcagctaacgcattaatggccccgcctggggagtacggccgcaaggctaaaactcaaaggaattgacgggggcccgcacaagcggcggagcatgtggcttaattcgacgcaacgcgaagaaccttaccaaggcttgacatacaccggaaacanccagagatggntgcccccttgtggtcggtgtacaggtggtgcatggctgtcgtcagctcgtgtcgtgagatgttgggttaagtcccgcaacgagcgcaacccttgntcccgtgttgccagcacgcccctcggggtggtggggactcacgggagaccgccggggtcaactcggaggaaggtggggacgacgtcaagtcatcatgccccttatgtcttgggctgcacacgtgctacaatggccggtacaatgagctgcgataccgcgaggtggagcgaatctcaaaaagccggtctcagttcggattggggtctgcaactcgaccccatgaagtcggagtcgctagtaatcgcggatcagcatgcca

(19) United States
(12) Patent Application Publication
(10) Pub. No.: US 2007/0259413 A1
(43) Pub. Date: Nov. 8, 2007

(54) Method of Decomposing Polyhydroxyalkanoate Resin

(76) Inventor: Yutaka Tokiwa, Ibaraki (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/578,331

(22) PCT Filed: Nov. 5, 2004
(86) PCT No.: PCT/JP04/16436

§ 371 Date: May 4, 2006

(30) Foreign Application Priority Data
Nov. 5, 2003 (JP) 2003-376269

(51) Int. Cl. C12P 1/06; C12N 9/16

(52) U.S. Cl. 435/169; 435/196

(57) ABSTRACT

A novel microorganism that directly and biologically degrades a polyhydroxyalkanoate resin and plastic containing the resin and a method therefor are provided. A method for degrading the polyhydroxyalkanoate resin, which comprises degrading the resin using a thermophile belonging to the genus Streptomyces, is provided.

### TECHNICAL FIELD

The present invention relates to a method for degrading polyhydroxyalkanoate with the use of a novel biological degradation method.

### BACKGROUND ART

Recently, plastic waste disposal has become problematic. Plastic waste disposal is mainly conducted by incineration or landfill. Incineration is problematic in that it promotes global warming, and landfill is problematic for reasons such as the reduction of ground area that can be used therefor. Thus, biological degradation methods are attracting attention. Polyhydroxyalkanoate resin has biodegradability, so that various applications of this resin as a next-generation plastic have been developed. In the near future, waste issues concerning such polyhydroxyalkanoate resin will surely emerge similarly to the situation of plastics that are currently used.

Polyhydroxyalkanoate is a polymer that is hydrolyzed in soil or in a water system. Polyhydroxyalkanoate has been noted as an alternative biodegradable plastic material for general purpose plastic that is difficult to be environmentally degraded. Examples of such polyhydroxyalkanoate resin include, depending on its constitutive monomer types, polyhydroxybutyrate, polyhydroxyvalerate, polyhydroxyhexanoate, polyhydroxyheptanoate, polyhydroxyoctanoate, polyhydroxynonanoate, polyhydroxydecanoate, and copolymers thereof.

Polyhydroxybutyrate is a type of polyhydroxyalkanoate that is known to be synthesized and stored within cells as an energy source for various microorganisms (see Non-patent documents 1 and 2). Furthermore, polyhydroxybutyrate is also known to be degraded by aerobic microorganisms (Non-patent documents 3 to 5) or anaerobic microorganisms (Patent document 1 and Non-patent documents 6 and 7).

Patent document 1: JP Patent No. 2889953

Non-patent document 1: “Journal of Bacteriology,” (U.S.A.) 1965, vol. 90, pp. 1455-1466

Non-patent document 2: “Macromolecule Bioscience,” 2001, vol. 1, pp. 1-24

Non-patent document 3: “Journal of Environmental Polymer Degradation,” (U.S.A.) 1993, vol. 1, pp. 53-63

Non-patent document 4: “Journal of Environmental Polymer Degradation,” (U.S.A.) 1993, vol. 1, pp. 227-233

Non-patent document 5: “Archives of Microbiology,” (Germany) 1996, vol. 154, pp. 253-259

Non-patent document 6: “Polymer Symposium” 1993, vol. 50, pp. 745-746

### DISCLOSURE OF THE INVENTION

### Objects to be Achieved by the Invention

Most degradation reactions of polyhydroxyalkanoate that have been reported so far are limited to those performed at a temperature between 25° C. and 30° C. It cannot be said that technology for aggressive degradation of such polyhydroxyalkanoate resin under high-temperature conditions, such as those pertaining in compost, has been sufficiently studied. Hence, objects of the present invention are to provide a novel microorganism capable of degrading such polyhydroxyalkanoate resin and plastic containing such resin at around 40° C. to 60° C. and to provide a method therefor.

### Means to Solve the Problems

As a result of wide-ranging screening and intensive studies to achieve the above objects, the present inventors have discovered an actinomycete of the genus Streptomyces having good activity of degrading polyhydroxyalkanoate and an enzyme (having the same activity) produced by actinomycete through microbial means. Thus, the present inventors have completed the present invention.

Specifically, the present invention provides the following (1) to (9):
(1) an enzyme derived from an actinomycete of the genus Streptomyces, which is capable of degrading a polyhydroxyalkanoate resin, having a molecular weight between approximately 47,000 to 56,000, having an optimum pH between 4 and 10, and having an optimum temperature between 40° C. and 55° C.;
(2) the enzyme according to (1) above, which is inductively produced by polyhydroxyalkanoate, hydroxybutyric acid, polyhydroxybutyrate, and/or hydroxybutyric acid ester;
(3) the enzyme according to (1) or (2) above, wherein the actinomycete of the genus Streptomyces is Streptomyces thermovulgaris, Streptomyces thermoolivaceus, Streptomyces thermohygroscopicus, or Streptomyces thermocarboxydovorans;
(4) the enzyme according to (1) or (2) above, wherein the actinomycete of the genus Streptomyces is a microorganism deposited under accession No. FERM P-19578;
(5) a method for degrading a polyhydroxyalkanoate resin, which comprises causing the polyhydroxyalkanoate resin to come into contact with the enzyme according to any one of (1) to (4) above so as to cause the resin to react with the enzyme;
(6) a method for degrading a polyhydroxyalkanoate resin, which comprises causing the polyhydroxyalkanoate resin to come into contact with an actinomycete of the genus Streptomyces so as to cause the resin to react with the actinomycte at 40° C. to 55° C.;
(7) the method according to (6) above, wherein the actinomycete of the genus Streptomyces is Streptomyces thermovulgaris, Streptomyces thermoolivaceus, Streptomyces thermohygroscopicus, or Streptomyces thermocarboxydovorans;
(8) the method according to (6) above, wherein the actinomycete of the genus Streptomyces is a microorganism deposited under accession No. FERM P-19578; and
(9) an actinomycete of the genus Streptomyces, which is capable of degrading a polyhydroxyalkanoate resin and is a microorganism deposited under accession No. FERM P-19578.

### EFFECT OF THE INVENTION

The method for degrading a polyhydroxyalkanoate resin of the present invention is a method for treating such polyhydroxyalkanoate resin and waste containing the same. This method is based on technology that can be performed without generating any exhaust gas, such as that generated in the cases of conventional incineration disposal methods. Furthermore, the method is extremely time-saving compared with landfill disposal. Thus, the method is very valuable for waste disposal. Specifically, a more environmentally beneficial method for degrading the polyhydroxyalkanoate resin, which is biodegradable plastic, involves not simply waiting for natural degradation of the resin in soil, but aggressively degrading the resin with the use of a microorganism or an enzyme capable of degrading such resin. The use of the degradation method of the present invention at a facility for making compost also enables conversion of the polyhydroxyalkanoate resin into useful substances such as organic acid or compost. Furthermore, the use of the method of the present invention facilitates recycling of the polyhydroxyalkanoate resin.

This description includes part or all of the contents as disclosed in the description and/or drawings of Japanese Patent Application No. 2003-376263, which is a priority document of the present application.

### BRIEF DESCRIPTION OF THE DRAWINGS

FIG. 1 shows changes with time concerning degradation of polyhydroxybutyrate resin powder by the MG2 strain. “●” and “∘” denote residual PHB levels (mg), “▪” and “□” denote water-soluble total organic carbon levels (TOC, mg), “♦” denotes dry cell weight (mg), and “∘” and “□” denote the results of control samples.

FIG. 2 shows the pH stability of the enzyme of the present invention.

FIG. 3 shows the temperature stability of the enzyme of the present invention. Each symbol denotes the preincubation time at each temperature (▪: 15 minutes, ●: 30 minutes, ♦: 45 minutes, and ▴: 60 minutes).

### BEST MODE OF CARRYING OUT THE INVENTION

The present invention provides a novel enzyme derived from an actinomycete of the genus Streptomyces, which is capable of degrading a polyhydroxyalkanoate resin. The enzyme of the present invention has a molecular weight between approximately 47,000 and 56,000, an optimum pH between 4 and 10, and preferably between 7 and 8, and an optimum temperature between 40° C. and 55° C. Furthermore, it has been discovered that the enzyme of the present invention is inductively produced in the presence of polyhydroxyalkanoate, hydroxybutyric acid, polyhydroxybutyrate, and/or hydroxybutyric acid ester.

The term “polyhydroxyalkanoate resin” in the present invention means a polymer of polyhydroxybutyric acid, polyhydroxyvaleric acid, polyhydroxyhexanoic acid, polyhydroxyheptanic acid, polyhydroxyoctanoic acid, polyhydroxynonanoic acid, polyhydroxydecanoic acid, or a copolymer thereof. Furthermore, examples of such polyhydroxyalkanoate resin include a polyhydroxy acid copolymer obtained through copolymerization of γ-butyrolactone with another component (e.g., β-propiolactone, β-butyrolactone, β-butyrolactone, ε-caprolactone, or ω-pentadecalactone) using a chemical catalyzer, a product obtained through blending of the above polymers, and a product obtained through blending of the above polymer with another component polymer, wherein the weight percentage of the hydroxyalkanoate component in each polymer is 10% or greater. Furthermore, the number average molecular weight of polyhydroxyalkanoate to which the degradation method of the present invention can be applied is approximately between 10,000 and 10⁶ and preferably approximately between 50,000 and 300,000. The present invention is not particularly limited to the aforementioned examples. Examples of known commercially available forms of such polyhydroxyalkanoate include polyhydroxybutyrate and a copolymer of polyhydroxybutyrate and polyhydroxyvalerate (produced by Sigma-Aldrich, Inc.). However, the method of the present invention is not limited to the use thereof.

The enzyme of the present invention can be obtained from an actinomycete of the genus Streptomyces, such as Streptomyces thermovulgaris, Streptomyces thermoolivaceus, Streptomyces thermohygroscopicus, or Streptomyces thermocarboxydovorans.

The above actinomycetes of the genus Streptomyces are conserved and available at facilities for microbial culture collection such as The Institute of Physical and Chemical Research (2-1 Hirosawa, Wako-shi, Saitama, Japan). In the present invention, one bacterial strain or a plurality of bacterial strains are preferably selected from Streptomyces thermovulgaris (JCM4338), Streptomyces thermoolivaceus strain (JCM492 1), Streptomyces thermohygroscopicus (JCM49 17), and Streptomyces thermocarboxydovorans (JCM 10367) and used. However, the strains used in the present invention are not particularly limited to these strains.

The present inventors have also discovered a novel actinomycete of the genus Streptomyces that produces the above enzyme from soil. This novel actinomycete of the genus Streptomyces was obtained as follows. Polyhydroxybutyrate (number average molecular weight (Mn) of 2.1×10⁵) was dispersed on each agar medium. Soil (collected at Tsukuba-shi) was placed on a plate containing such agar medium, and culture was then conducted at 50° C. The novel actinomycete was obtained from bacteria that had formed clear zones. From among strains confirmed to have degradation activity, a strain that had formed obvious clear zones within 24 hours of culture and had particularly high activity was named the MG2 strain. The MG2 strain was deposited internationally under the regulations of the Budapest Treaty with the International Patent Organism Depositary of the National Institute of Advanced Industrial Science and Technology (Tsukuba Central 6, 1-1-1 Higashi, Tsukuba, Ibaraki, Japan) as of Nov. 4, 2003 under accession No. FERM P-19578 (FERM ABP-10158). The above actinomycete strain (MG2) could be cultured under a temperature between 25° C. and 60° C. and showed good proliferation particularly at 50° C. As a result of phylogenetic analysis, it was discovered that the MG2 strain is a novel bacterial species showing 97.0% and 96.5% sequence similarlity with Streptomyces thermocarboxydovorans and Streptomyces thermodiastaticus, respectively.

The above actinomycete of the genus Streptomyces was revealed to be able to secrete the above enzyme of the present invention in media (outside bacterial bodies). The enzyme of the present invention has a molecular weight between approximately 47,000 and 56,000, as analyzed by SDS-PAGE electrophoresis.

The present invention also provides a method for degrading a polyhydroxyalkanoate resin, which comprises causing the polyhydroxyalkanoate resin to come into contact with the above enzyme of the present invention so as to cause the resin to react with the enzyme.

The present invention also provides a method for degrading a polyhydroxyalkanoate resin, which comprises causing the polyhydroxyalkanoate resin to come into contact with an actinomycete of the genus Streptomyces, so as to cause the resin to react with the actinomycete at a temperature between 40° C. and 55° C.

The method of the present invention enables degradation of a polyhydroxyalkanoate resin, whereby degradation is performed using an actinomycete of the genus Streptomyces or the enzyme derived from such actinomycete.

Examples of a preferable actinomycete of the genus Streptomyces that is used in the method of the present invention include Streptomyces thermovulgaris, Streptomyces thermoolivaceus, Streptomyces thermohygroscopicus, and Streptomyces thermocarboxydovorans.

A further preferable actinomycete of the genus Streptomyces that is used in the method of the present invention is an actinomycete belonging to the bacterial strain that was internationally deposited under accession No. FERM P-19578.

An example of a particularly preferable actinomycete is the MG2 strain that the present inventors have deposited under accession No. FERM P-19578.

The method of the present invention comprises incubating polyhydroxyalkanoate with the actinomycete, the above MG2 strain of the genus Streptomyces, the Streptomyces thermovulgaris strain, the Streptomyces thermoolivaceus strain, the Streptomyces thermohygroscopicus strain, or the Streptomyces thermocarboxydovorans strain or enzymes derived from such actinomycetes in a medium containing inorganic salts and thus degrading the polyhydroxyalkanoate.

The above bacterial strains may be used in the form of a liquid for degradation of the polyhydroxyalkanoate resin. Specifically, such culture solution is prepared by culturing and growing the above bacterial strains in a basic medium appropriate for their culture, such as a nitrogen-source-containing inorganic salts medium supplemented with 50 ppm to 500 ppm yeast extract. If necessary, the bacterial bodies of the above bacterial strains may be used for degradation of the polyhydroxyalkanoate resin in the form of freeze-dried powder (freeze-dried by a standard method) or in the form of solid preparations such as tablets that are prepared by blending the thus obtained powders with various vitamins or minerals and necessary nutrition sources (e.g., yeast extract, casamino acid, or peptone) followed by tabletting of the resultant.

A basic medium that is used for culture in the present invention contains inorganic salts. Examples of a culture source that is generally used in such medium include a nitrogen source such as ammonium sulfate, ammonium phosphate, or ammonium carbonate and another inorganic salt such as monopotassium phosphate, dipotassium phosphate, magnesium sulfate, sodium chloride, ferrous sulfate, sodium molybdate, sodium tungstate, or manganese sulfate. Unlike a general medium for microorganisms, addition of a small amount of yeast extract, casamino acid, peptone, malt extract, or the like may be effective. Furthermore, Plysurf (DAI-ICHI KOGYO SEIYAKU CO., LTD.), which is a surfactant, or octylglucoside may also be used to disperse powdery polyhydroxyalkanoate. Furthermore, to induce the production of an enzyme capable of degrading the polyhydroxyalkanoate resin, it is effective to add polyhydroxyalkanoate, hydroxybutyric acid, polyhydroxybutyrate, or hydroxybutyric acid ester to a medium. The pH for a medium is between 4.0 and 10.0, and preferably between 5.0 and 8.0. Temperature for culture is between 25° C. and 70° C., and preferably between 40° C. and 55° C.

When the polyhydroxyalkanoate resin is degraded according to the method of the present invention by an enzyme produced by an actinomycete of the genus Streptomyces, such as the MG2 strain, the Streptomyces thermovulgaris strain, the Streptomyces thermoolivaceus strain, the Streptomyces thermohygroscopicus strain, or the Streptomyces thermocarboxydovorans strain, such enzyme can be used with a pH regulator, a stabilizer, an excipient, a surfactant, or the like that is appropriately added.

The biological degradation method for a polyhydroxyalkanoate resin of the present invention is preferably performed with the addition of one of the above described basic media, a polyhydroxyalkanoate resin to be treated, and one of the above bacterial strain or powders, tablets, or a culture solution containing such a bacterial strain blended therein to a culture vessel. The above bacterial strain may be incorporated into activated sludge or compost. It is most preferable to previously powder the polyhydroxyalkanoate resin in terms of degradation efficiency. The polyhydroxyalkanoate resin may be in the form of film. In addition, the amount of the polyhydroxyalkanoate resin to be added to a basic medium is preferably between 0.01 wt. % and 10 wt. %. Microbes may be added in a minimal amount. A preferable amount of microbes to be added to the polyhydroxyalkanoate resin is 0.01 wt. % or more, so that the amount to be added has no effects on the degradation time.

The time required for degradation differs depending on the composition, shape, and amount of the polyhydroxyalkanoate resin, the type of a microbe used, the relative amount of such microbe to an enzyme or the resin, and other various culture conditions, for example. Thus, the time required for degradation cannot be completely specified. Through several minutes, several weeks, or several months of maintenance of such conditions, the polyhydroxyalkanoate resin can be successfully degraded.

### EXAMPLES

The present invention will be further described specifically by referring to examples. However, the scope of the present invention is not limited by these examples.

### Example 1 Obtainment of a Novel Actinomycete of the Genus Streptomyces

Polyhydroxybutyrate (number average molecular weight (Mn) of 2.1×10⁵, “BIOGREEN” produced by MITSUBISHI GAS CHEMICAL COMPANY. INC.) was dispersed at 1% on an agar medium (a medium with the composition as shown in Table 2 that had been supplemented with agar) on a plate. Soil collected at Tsukuba-shi was placed on the plate, and culture was then conducted at 50° C. The novel actinomycte was obtained from among bacteria that had formed clear zones.

The biological characteristics of the thus obtained novel actinomycete of the genus Streptomyces are listed in Table 1.

TABLE 1
Biological characteristics of MG2 strain

| Characteristics | MG2 results |
|---|---|
| Morphology | Unequal, folded, and dark gray |
| Gram stain | + |
| Motility | − |
| Catalase | + |
| Oxidase | + |
| Fermentability in glucose OF^{a} | − (oxidation) |
| Growth | |
| 25° C. | − |
| 30° C. | + |
| 50° C. | + |
| 55° C. | + |
| 60° C. | − |
| Yeast Malt + 50 μ/ml novobiocin | + |
| Acid glucose peptone agar pH10 | + |
| Growth oatmeal agar pH 10 | + |
| Melanin pigment | − |
| Tyrosine | − |
| Xanthine | − |
| Elastin | + |
| Starch | + |
| Casein | + |
| Egg yolk agar | No growth |
| Esculin | + |
| Gelatin | + |

^{a}OF: oxidation fermentation solution

16S-rRNA was obtained from the thus obtained bacterial strain according to a standard method and then the sequence was specified. The sequence is shown in SEQ ID NO: 1.

As a result of comparison of this sequence with sequences of various conventionally known bacteria, this bacterium was found to have 97.0% sequence identity and 96.5% sequence identity with Streptomyces thermocarboxydovorans and Streptomyces thermodiastaticus, respectively. Thus, the strain was revealed to be a conventionally unknown novel actinomycete. The present inventors named the novel actinomycete the MG2 strain.

### Example 2 Activity of Degrading Polyhydroxybutyrate Resin Powder

Degradation of PHB powder by the MG2 strain obtained in Example 1 was examined.

100 ml of a basic medium having the composition shown in Table 2 below and 200 mg of PHB powder (number average molecular weight (Mn) of 2.1×10⁵; “BIOGREEN” produced by MITSUBISHI GAS CHEMICAL COMPANY. INC.; uniform particle size because the particles had passed through a 250 μm wire screen) were added to a 500 ml Erlenmeyer flask. 5 ml of a culture solution of the MG2 strain was added to the flask, and this was followed by incubation at 50° C. in a rotary shaker (180 rpm).

TABLE 2

| | Blending amount | |
|---|---|---|
| Components | (per liter of distilled water) | |
| Na₂MoO₄•H₂O | 0.5 | mg |
| Na₂WO₄•2H₂O | 0.5 | mg |
| FeSO₄•7H₂O | 10 | mg |
| CaCl₂•2H₂O | 20 | mg |
| NaCl | 100 | mg |
| MgSO₄•7H₂O | 200 | mg |
| (NH₄)₂SO₄ | 1000 | mg |
| K₂HPO₄ | 1600 | mg |
| KH₂PO₄ | 200 | mg |
| Yeast extract | 100 | mg |

The medium was subjected to extraction using chloroform every 12 hours. Thus, undegraded polymer was collected and then dried using a rotary evaporator. Furthermore, cells were also collected, centrifuged, and then dried. Water-soluble organic carbon (TOC) was measured using a TOC-5000 analyzer (Shimadzu Corporation). Furthermore, HPLC using a sulfonate polystyrene gel column was performed using 4 mM perchloric acid as a mobile phase (0.6 ml/minute), and the degradation product was analyzed at 40° C. At the same time, D-3 hydroxybutyric acid was detected using an enzymatic bioanalysis kit (Boehringer Mannheim/R-Biopharm).

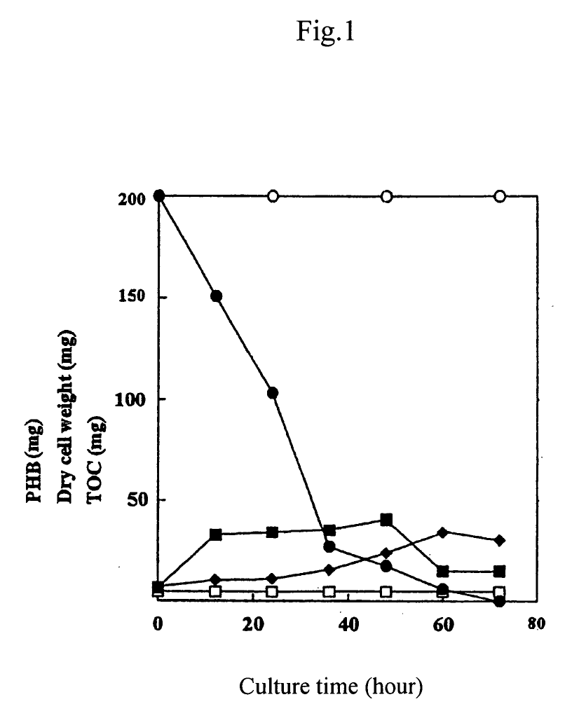

As a result, through 24 hours of incubation, degradation of PHB powder by 50% was observed, in addition to accumulation of water-soluble total organic carbon (TOC) in the cells (FIG. 1). In the culture solution, accumulation of D-3 hydroxybutyric acid, adipic acid, and succinic acid was detected. The PHB powder was completely degraded through 3 days of incubation.

### Example 3 Degradation of Polyhydroxybutyrate Film

Degradation of PHB film (cast film prepared using “BIOGREEN” produced by MITSUBISHI GAS CHEMICAL COMPANY. INC.) by the MG2 strain obtained in Example 1 was examined using a scanning electron microscope (JEOL model JSM-T220, 15 kV).

Incubation was performed under conditions similar to those in Example 2. Because of the action of cells that had adhered to the film, dome-shaped holes were formed on the film. The film was completely degraded after 6 days of incubation.

### Example 4 Degradation Activity of Various Actinomycetes of the Genus Streptomyces

Polyhydroxybutyrate (number average molecular weight (Mn) of 2.1×10⁵) was dispersed on an agar medium on a plate. Various actinomycete strains of the genus Streptomycin were each inoculated on the plate and then cultured at 50° C. The degrees of clear zone formation are: +++(formation within 1 to 2 days), ++(formation within 5 to 7 days), +(formation within 8 to 14 days), and −(no formation).

TABLE 3

| Strain | Reaction temperature | Clear zone |
|---|---|---|
| (JCM registration No.) | (° C.) | formation |
| MG2 | 50 | +++ |
| S. thermovulgaris (4338) | 50 | +++ |
| S. thermoolivaceus (4921) | 50 | ++ |
| S. thermophilus (4336) | 50 | − |
| S. thermoviolaceous (4337) | 50 | − |
| S. thermohygroscopicus | 45 | +++ |
| (4917) | | |
| S. thermocarboxydovorans | 45 | +++ |
| (10367) | | |
| S. thermodiastaticus (4314) | 45 | − |
| S. thermodiastaticus (4840) | 45 | − |

As is clear from the results in Table 3, obvious clear zones were formed by the MG2 strain obtained in Example 1, Streptomyces thermovulgaris, Streptomyces thermoolivaceus, Streptomyces thermohygroscopicus, and Streptomyces thermocarboxydovorans. It was confirmed that these strains have activity of degrading polyhydroxybutyrate.

### Example 5 Degradation Activity of Various Actinomycetes of the Genus Streptomyces

200 mg of polyhydroxybutyrate (powdered at the size of 180 microns or less; the number average molecular weight (Mn) of 2.1×10⁵) was added as a carbon source to 100 ml of a medium (pH 7.0) with the composition shown in Table 4. Each bacterial strain listed in Table 5 was inoculated on each medium, and this was followed by 4 days of culture at 50° C. using a 180-rpm rotational shaker.

After degradation of powdered polyhydroxybutyrate that had been added, water-soluble total organic carbon (TOC, ppm) levels were calculated. The results are listed in Table 5. In cases in which bacteria having the degradation ability according to the present invention were added, the thus generated total organic carbon levels ranged from 16 ppm to 937 ppm.

TABLE 4

| | Blending amount | |
|---|---|---|
| Components | (per liter of distilled water) | |
| NaMoO₄•H₂O | 0.5 | mg |
| Na₂WO₄•2H₂O | 0.5 | mg |
| FeSO₄•7H₂O | 10 | mg |
| CaCl₂•2H₂O | 100 | mg |
| NaCl | 200 | mg |
| MgSO₄ | 1000 | mg |
| (NH₄)₂SO₄ | 1600 | mg |
| K₂HPO₄ | 200 | mg |
| KH₂PO₄ | 100 | mg |
| Yeast extract | 200 | mg |

TABLE 5

| | | Total dissolved organic |
|---|---|---|
| Bacterial strain | Temperature (° C.) | carbon level (ppm) |
| MG2 strain | 50 | 937 |
| Streptomyces | 50 | 13 |
| thermovulgaris strain | | |
| (JCM4338) | | |
| Streptomyces | 50 | 16 |
| thermoolivaceus strain | | |
| (JCM4921) | | |
| Streptomyces | 45 | 123 |
| thermohygroscopicus | | |
| (JCM4917) | | |
| Streptomyces | 45 | 16 |
| thermocarboxydovorans | | |
| (JCM10367) | | |

As described above, it was confirmed that the MG2 strain, an actinomycete of the genus Streptomyces, the Streptomyces thermovulgaris strain, the Streptomyces thermoolivaceus strain, the Streptomyces thermohygroscopicus strain, and the Streptomyces thermocarboxydovorans strain can degrade polyhydroxybutyrate, which is a polymer.

### Example 6 pH Stability of Enzyme Activity

The pH stability of the enzyme activity of degrading polyhydroxyalkanoate was examined using the MG2 strain obtained in Example 1.

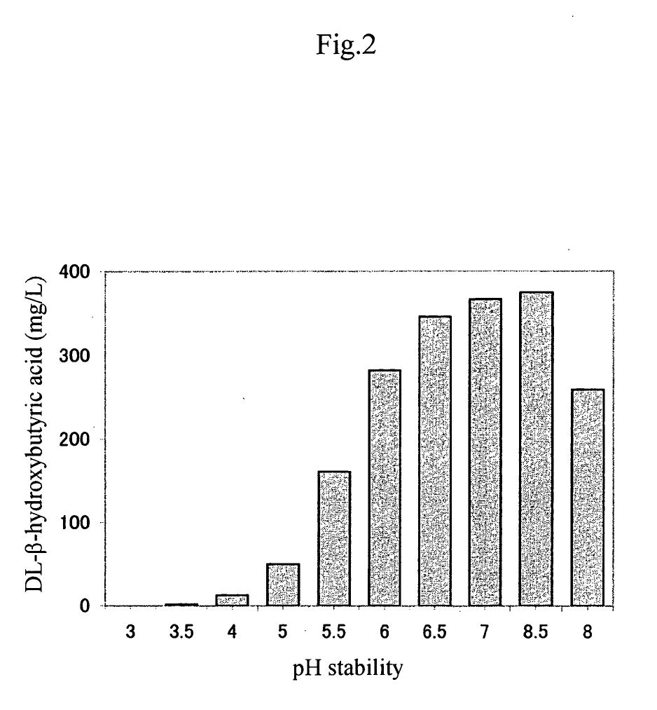

The MG2 strain was added to 100 ml of a medium containing 100 mg of PHB powder and-having the composition shown in Table 4 and then cultured with shaking for 48 hours at 50° C. After filtration, the filtered solution was subjected to dialysis against a 0.01 M phosphate buffer (pH 7.0). Buffers with various pHs (e.g., 3.9 ml of a citrate buffer (0.1 M) with pH 3.0, 3.5, 4.0, or 5.0 and 3.9 ml of a phosphate buffer (0.1 M) with pH 5.5, 6.0, 6.5, 7.0, 7.5, or 8.0) were added to the thus dialyzed enzyme solutions (1 ml each). After 24 hours of incubation at 5° C., reaction was performed at 50° C. for 16 hours in a buffer containing 10 mg of PHB and 0.5% (w/v) octylglucopyranoside. Water-soluble total organic carbon and DL-hydroxybutyric acid sodium salt levels in each medium were measured in a manner similar to that in Example 2. The results of detecting DL-hydroxybutyric acid sodium salt are shown in FIG. 2.

As shown in the results in FIG. 2, it was confirmed that the enzyme of the present invention was stable within a pH range between 4 and 10. In particular, the enzyme can maintain its high activity within a pH range between 5 and 8.

### Example 7 Temperature Stability of Enzyme Activity

The temperature stability of the MG2 strain's enzyme activity of degrading polyhydroxyalkanoate was examined.

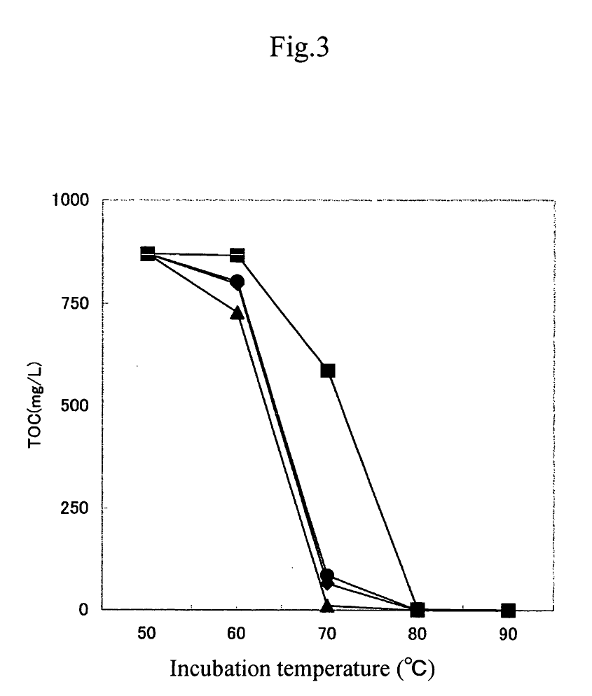

The bacterial strain was cultured for 48 hours. 5 ml of a medium was sampled and then incubated with shaking at 50° C., 60° C., 70° C., 80° C., or 90° C. for 60 minutes. After 4 hours of incubation at 50° C., the water-soluble total organic carbon level in each medium was measured. The reaction mixture was composed of: 10 mg of PHB powder, 0.1 M phosphate buffer (pH 7.0), 0.1 ml of 0.5% (w/v) octylglucopyranoside, and 1 ml of a medium obtained by filtering bacterial bodies through a Millipore filter with a pore size of 0.2 μm. FIG. 3 shows the results.

Based on these results, it was confirmed that the enzyme of the present invention is stable within a temperature range up to 60° C.

### Example 8 Induction of Degradation Activity Using Substrate

Whether or not the induction of enzyme activity was observed in the presence of various reaction substrates was examined.

In the presence of various substrates listed in Table 6 and Table 7, water-soluble total organic carbon (TOC) generation and hydroxybutyric acid generation were detected. The composition of the reaction mixture was the same as that in Example 6. Reaction was performed at 50° C. for 16 hours. As a result, as shown in Table 6 and Table 7, significant increases were detected in soluble TOC levels and hydroxybutyric acid levels in the presence of polyhydroxyalkanoate, hydroxybutyric acid, polyhydroxybutyrate, and/or hydroxybutyric acid ester.

TABLE 6

| | Water-soluble | Hydroxybutyric |
|---|---|---|
| | TOC | acid |
| Substrate | generation (mg/L) | generation (mg/L) |
| PHB | 926 | 879 |
| (R)-(−)-3-sodium-3- | 731 | 820 |
| hydrdoxybutyric acid | | |
| L-(+)-β-hydroxybutyric acid | 8 | 4.8 |
| DL-3-hydroxy-n-sodium butyrate | 306 | 269 |
| DL-3-hydroxy-n-butyric acid ethyl | 618 | 536 |
| ester | | |
| (R)-(−)-3-hydroxy-n-butyric acid | 1394 | 943 |
| methyl ester | | |
| (S)-(+)-3-hydroxy-n-butyric acid | 79 | 69 |
| methyl ester | | |
| (S)-(+)-3-hydroxy-n-butyric acid | 69 | 66 |
| ethyl ester | | |
| β-hydroxyisovaleric acid | — | 2.98 |
| (3-hydroxy-3-methylbutyrate) | | |
| β-butyrolactone | — | 3.6 |
| (β-methylpropiolactone) | | |

TABLE 7

| | Water-soluble TOC |
|---|---|
| Substrate | generation (mg/L) |
| PHB | 622 |
| Glucose | 17 |
| Sucrose | 5 |
| Lactose | 6 |
| Maltose | 3 |
| Starch | 20 |
| Glycerol | 12 |

### Reference Example 1

The presence or the absence of activity of degrading various polymers was examined using the MG2 strain obtained in Example 1 in a manner similar to that in Example 4.

Polymer powders used herein were: poly(ethylene succinate) (PES; number average molecular weight 5.92×10⁴; produced by NIPPON SHOKUBAI CO., LTD.), poly(ester carbonate) (PEC; carbonate content of 18 mol %; number average molecular weight of 2.4×10⁵; produced by MITSUBISHI GAS CHEMICAL COMPANY. INC.), polycaprolactone (PCL; trade name: Tone P-767; number average molecular weight of 6.73×10⁴; produced by Union Carbide Corporation), poly(butylene succinate) (PBS; trade name: Bionolle 1020; number average molecular weight of 4.8×10⁴; produced by SHOWA HIGH POLYMER CO., LTD.), and poly(L-lactide) (PLA; trade name: Lacty 1012; number average molecular weight of 1.88×10⁵; produced by Shimadzu Corporation). The MG2 strain was cultured at 50° C. on each agar medium containing each polymer. Table 8 shows the results.

TABLE 8

| Polymer | Clear zone formation |
|---|---|
| Poly(3-hydroxybutyrate) | +++ |
| Poly(ethylene succinate) | ++ |
| Poly(ester carbonate) | ++ |
| Polycaprolactone | + |
| Poly(butylene succinate) | + |
| Poly(L-lactide) | − |

As is clear from the results in Table 8, not only the polyhydroxybutyrate resin, but also polyethylene succinate and polyester carbonate were degraded through contact with the MG2 strain. Furthermore, it was revealed that polycaprolactone and polybutylene succinate were also degraded, although the degradation activity was weak. Such tendency was similarly observed in a case where a culture solution of the MG2 strain had been used and accumulation of water-soluble degradation products had been measured.

All publications, patents, and patent applications cited herein are incorporated herein by reference in their entirety.

1. An enzyme derived from an actinomycete of the genus Streptomyces, which is capable of degrading a polyhydroxyalkanoate resin, having a molecular weight between approximately 47,000 to 56,000, having an optimum pH between 4 and 10, and having an optimum temperature between 40° C. and 55° C.

2. The enzyme according to claim 1, which is inductively produced by polyhydroxyalkanoate, hydroxybutyric acid, polyhydroxybutyrate, and/or hydroxybutyric acid ester.

3. The enzyme according to claim 1 or 2, wherein the actinomycete of the genus Streptomyces is Streptomyces thermovulgaris, Streptomyces thermoolivaceus, Streptomyces thermohygroscopicus, or Streptomyces thermocarboxydovorans.

4. The enzyme according to claim 1 or 2, wherein the actinomycete of the genus Streptomyces is a microorganism deposited under accession No. FERM BP-10158.

5. A method for degrading a polyhydroxyalkanoate resin, which comprises causing the polyhydroxyalkanoate resin to come into contact with the enzyme according to claim 1 so as to cause the resin to react with the enzyme.

6. A method for degrading a polyhydroxyalkanoate resin, which comprises causing the polyhydroxyalkanoate resin to come into contact with an actinomycete of the genus Streptomyces so as to cause the resin to react with the actinomycete at 40° C. to 55° C.

7. The method according to claim 6, wherein the actinomycete of the genus Streptomyces is Streptomyces thermovulgaris, Streptomyces thermoolivaceus, Streptomyces thermohygroscopicus, or Streptomyces thermocarboxydovorans.

8. The method according to claim 6, wherein the actinomycete of the genus Streptomyces is a microorganism deposited under accession No. FERM BP-10158.

9. An actinomycete of the genus Streptomyces, which is capable of degrading a polyhydroxyalkanoate resin and is a microorganism deposited under accession No. FERM BP-10158.
